(19) United States
(12) Patent Application Publication
(10) Pub. No.: US 2003/0158249 A1
(43) Pub. Date: Aug. 21, 2003

(54) Use of docetaxel for treating hepatoma

(75) Inventors: Chin-Wen Chi, Taipei (TW); Heng-Liang Lin, Taipei (TW); Tsung-Yun Liu, Taipei (TW); Wing-Yiu Lui, Taipei (TW); Gar-Yang Chau, Taipei (TW)

Correspondence Address: Finnegan, Henderson, Farabow,, Garrett & Dunner, L.L.P., 1300 I Street, N.W., Washington, DC, 20005-3315, US

(73) Assignee: Aventis Pharma S.A.

(21) Appl. No.: 10/083,565

(22) Filed: Feb. 27, 2002

Related U.S. Application Data
(63) Continuation of application No. PCT/EP00/08782, filed on Aug. 29, 2000.

(30) Foreign Application Priority Data
Aug. 31, 1999 (GB) 99 20548.6

(51) Int. Cl. A61K 31/337

(52) U.S. Cl. 514/449

(57) ABSTRACT

The present invention is based on the finding that docetaxel is significantly more active against hepatocellular carcinoma cells than paclitaxel at concentrations of up to 1 μM. It accordingly provides the use of docetaxel, or a hydrate thereof, in the manufacture of a medicament for use in the treatment of hepatocellular carcinoma.

This invention relates to treatment of hepatocellular carcinoma.

Hepatocellular carcinoma (HCC) is one of the most common cancers in Southeast Asia and African countries. In Taiwan, HCC is the leading cause of death in male cancer patients. The survival rate of HCC patients is very low. This is mainly due to lack of effective treatments. Irradiation and chemotherapies have not so far proved to be satisfactory; surgery is the most effective treatment for HCC. However, surgery is only appropriate for patients with small resectable tumours.

Recently, antimitotic drugs such as paclitaxel have received renewed interest. Paclitaxel was originally isolated from the bark of the Yew tree. The antitumour effect of paclitaxel has been known since 1971. Paclitaxel inhibits tumour cell division by its action on microtubule assembly. In vitro analyses using tumour cells have revealed that paclitaxel arrests cells mainly in the G2/M phase of the cell cycle (Schiff PB and Horwitz SB, Proc. Natl. Acad. Sci 77, 1561-1565, 1980). Recent studies have shown that paclitaxel is effective against various malignant tumour cells such as brain tumour, gastric and prostate cancer, breast cancer, melanoma and ovarian cancer.

However, paclitaxel is not effective against hepatocellular carcinoma. A phase II clinical trial of paclitaxel for HCC patients is reported in British Journal of Cancer, 78 (1), 34-39, 1998. That article concludes that paclitaxel had no significant anti-cancer effect in HCC patients.

As explained above, the cytotoxic effect of paclitaxel has been found to be cell cycle dependent, with cell cycle arrest occurring mainly at the G2/M phase. However, it has now been found that docetaxel can achieve non cell cycle dependent cytotoxicity in HCC cells. This indicates that the cytotoxic effect of docetaxel on HCC cells is achieved by a different mechanism from that of paclitaxel. Further, the in vitro activity of docetaxel against HCC cells is significantly higher than that of paclitaxel at concentrations of up to 1 μM. Given the highly cytotoxic nature of the taxoids, an increased activity at low concentration suggests that docetaxel, unlike paclitaxel, will be of practical use in the clinical treatment of hepatocellular carcinoma.

Accordingly, the present invention provides the use of docetaxel or a hydrate thereof in the manufacture of a medicament for use in the treatment of hepatocellular carcinoma.

Also provided is a method of treating a patient suffering from hepatocellular carcinoma, which method comprises administering to said patient an effective amount of docetaxel or a hydrate thereof. The invention also provides a method for ameliorating the condition of a patient suffering from hepatocellular carcinoma, which method comprises administering to said patient an effective amount of docetaxel or a hydrate thereof.

Docetaxel is a known compound. It has the formula

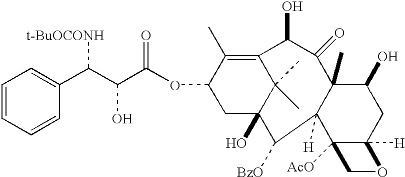

Processes for the preparation of docetaxel are described in EP-A-253738 and EP-A-336841.

Docetaxel may be used, for example, in anhydrous form or as a hydrate. As used herein, references to docetaxel include references to hydrates thereof.

Docetaxel hydrates can be prepared by dissolving anhydrous docetaxel in an organic solvent such as acetone, ethanol, acetonitrile or N,N-dimethylformamide, and by recrystallising docetaxel hydrate by adding the thus obtained solution to water. A docetaxel hydrate is typically a dihydrate, a trihydrate or a tetrahydrate. In particular, the trihydrate has been found to be particularly stable, and docetaxel trihydrate is accordingly preferred. Docetaxel trihydrate may be prepared by the processes set out in EP-A-770070.

Docetaxel is unexpectedly active against hepatocellular carcinomas. In particular, it can be used to treat liver cell carcinomas, fibrolamellar variants and mixed hepatocellular cholangiocarcinomas.

In the present invention, docetaxel may be administered by any conventional route known for the administration of docetaxel. Thus, it may, for example, be administered parenterally. Typically, it is administered intravenously, preferably by intravenous infusion.

In the present invention, docetaxel is typically formulated for administration as a pharmaceutically acceptable composition containing docetaxel and a pharmaceutically acceptable carrier or diluent. Suitable carriers and diluents include non-toxic solvents and suspension media, for example sterile aqueous media. Preferably, the compositions take the form of aqueous solutions or suspensions, for example, solutions suitable for injection or infusion, which can contain emulsifying agents, colourings, preservatives or stabilizers.

Pharmaceutical compositions suitable for parenteral administration include sterile aqueous or non-aqueous solutions or suspensions. Suitable sterile non-aqueous solutions and suspensions include solutions and suspensions in natural vegetable oils such as olive oil, sesame oil or liquid petroleum or in injectable organic esters such as ethyl oleate. Suitable sterile aqueous solutions include solutions of docetaxel in water. Typically, the pH of sterile aqueous solutions suitable for parenteral administration is appropriately adjusted. Further, such sterile aqueous solutions are generally made isotonic, for example with a sufficient amount of sodium chloride or glucose. It is particularly preferred that solutions suitable for administration by infusion have a pH similar to that of the blood and are made isotonic.

Sterilization may be carried out by heating or by any other means which does not adversely affect the composition.

Pharmaceutical compositions containing docetaxel suitable for use in the present invention may further comprise a surfactant. Preferred surfactants are polysorbates, polyoxyethylene glycol esters and ester-ethers of polyethylene glycol and castor oils. Examples of suitable surfactants, and of pharmaceutical compositions containing the surfactants, can be found in AU-A-666859.

Docetaxel may also be formulated for use in the present invention as a lyophilized composition. Such lyophilized compositions have good physical and chemical stability and can therefore be stored for long periods. Lyophilised compositions containing docetaxel may be prepared by lyophilising an aqueous solution of docetaxel by standard techniques. They may further comprise bulking agents such as lactose. They may also comprise tonicity adjustment agents such as sugars and polymers. Examples of suitable tonicity adjustment agents include glucose, dextrose and mannitol and polymers, for example polyvinylpyrrolidone.

A lyophilized composition may be redissolved at the time of use in any compatible and pharmaceutically acceptable injectable medium. The lyophilizate may be advantageously taken up with injection grade double-distilled water, in a volume equivalent to the initial volume of the solution to be lyophilized.

A pharmaceutical composition containing docetaxel suitable for use in the present invention typically contains at least 0.01% by weight of therapeutically active product. Generally, a pharmaceutical composition contains from 0.01 to 1000 mg, preferably from 0.1 to 500 mg, of therapeutically active product.

Preferably, a solution suitable for intravenous injection contains from 38 to 42, more preferably around 40 mg/ml of active product. Typically, such solutions are provided in vials containing 20 mg or 80 mg of active product.

Preferably, a solution suitable for infusion contains from 0.1 to 11, preferably from 0.1 to 10, more preferably from 0.3 to 0.9 mg/ml of active product.

Therapeutic treatment with docetaxel according to the present invention may be performed concurrently with other therapeutic treatments including treatment with other antineoplastic drugs, monoclonal antibodies, immunotherapy or radiotherapy or biological response modifiers. Suitable biological response modifiers include lymphokines and cytokines such as interleukins, interferons (α, β or δ) and TNF. Other chemotherapeutic agents which are useful in the treatment of disorders due to abnormal cell proliferation include alkylating agents, for instance nitrogen mustards such as mechlorethamine, cyclophosphamide, melphalan and chlorambucil, alkyl sulphonates such as busulfan, nitrosoureas such as carmustine, lomustine, semustine and streptozocin, triazenes such as dacarbazine, antimetabolites such as folic acid analogues, for instance methotrexate, pyrimidine analogues such as fluorouracil and cytarabine, purine analogues such as mercaptopurine and thioguanine, natural products, for instance vinca alkaloids such as vinblastine, vincristine and vindesine, epipodophyllotoxins such as etoposide and teniposide, antibiotics such as dactinomycin, daunorubicin, doxorubicin, bleomycin, plicamycin and mitomycin, enzymes such as L-asparaginase, various agents such as coordination complexes of platinum, for instance cisplatin, substituted ureas such as hydroxyurea, methyl-hydrazine derivatives such as procarbazine, adrenocortical suppressants such as mitotane and aminoglutethimide, hormones and antagonists such as adrenocortico-steroids such as prednisone, progestins such as hydroxyprogesterone caproate, methoxyprogesterone acetate and megestrol acetate, oestrogens such as diethylstilbo-estrol and ethynyloestradiol, antioestrogens such as tamoxifen, and androgens such as testosterone propionate and fluoxymesterone.

Concurrent treatment with cyclophosphamide, 5-fluorouracil, etoposide, vinorelbine or methotrexate is preferred, as synegism between these compounds and docetaxel may be achieved. Further, 2-methoxyestradiol is active against hepatocellular carcinomas and has been found to be well tolerated after 1 month of daily treatment in mice (Klauber et al, Cancer Research, 57, 81-86, 1997). Concurrent treatment with 2-methoxyestradiol is therefore also preferred, particularly when chronic treatment is required.

In the present invention, docetaxel is administered at a dosage which permits the treatment of hepatocellular carcinoma. The dosage varies according to the route of administration and the physical characteristics of the patient. Suitable dosages include those which are therapeutically effective for the treatment of disorders due to abnormal cell proliferation. Docetaxel may be administered as often as necessary to obtain the desired therapeutic effect.

A typical dose of docetaxel for the treatment of a human is from 50 to 150, preferably 60 to 100, more preferably around 100 mg docetaxel/m² of surface area of the patient's skin. When docetaxel is administered by infusion, the rate of infusion is typically from 1 to 200, preferably around 100 mg/m² docetaxel per hour.

The above dose may be repeated as required. Typically, it is repeated daily or weekly. Preferably, it is repeated every 3 weeks. For example, docetaxel may be administered at a dose of around 100 mg/m² as an intravenous infusion over 1 hour every 3 weeks.

The following Example illustrates the invention.

### EXAMPLE

Materials and Methods

Unless otherwise indicated, the methods used are standard biochemical techniques. The cell lines used are all commercially available.

Cell Culture

The experiments detailed below involve human hepatoma cell lines Hep3B (ATCC designation HB 8064), HepG2 (ATCC designation HB 8065) and HA22T/VGH, and murine hepatoma cell line Hepa 1-6. These cells were cultured in DMEM (GIBCO, BRL) containing 10% fetal bovine serum (Hyclone), 0.01 mg/ml gentamycin and 0.1 mM non-essential amino acid. Cells were grown in a CO₂ incubator at 37° C., with 5% CO₂ and 95% filtered air.

Drug Treatment

In the experiments detailed below, the above hepatoma cells were treated with different concentrations of paclitaxel (0.001-10 pM) and docetaxel (0.001-10μM) for 24 hours and 72 hours. Paclitaxel was dissolved in dimethylsulfoxide (DMSO) and docetaxel was dissolved in ethanol as stock solutions. The final concentration of vehicle was less than 0.1%.

Cell Viability Study: MTT assay

Cells were cultured in a 96 well cell culture cluster (COSTAR) at a density of 4×10 cells/ml. After drug treatment for 24 hours or 72 hours, medium was discarded and replaced with an equal volume (100μl ) of fresh medium containing MTT (0.456 mg/ml; 3-[4,5-Dimethylthiazol-2-yl]-2,5-diphenyl-tetrazolium bromide) and incubated for 1.5 hours at 37° C. The fresh medium was then discarded, and 100 μl DMSO was then added. Cell viability was determined by colorimetric comparison by reading OD values from a microplate reader (SPECTRA MAX 250) at an absorption wavelength of 570 nm.

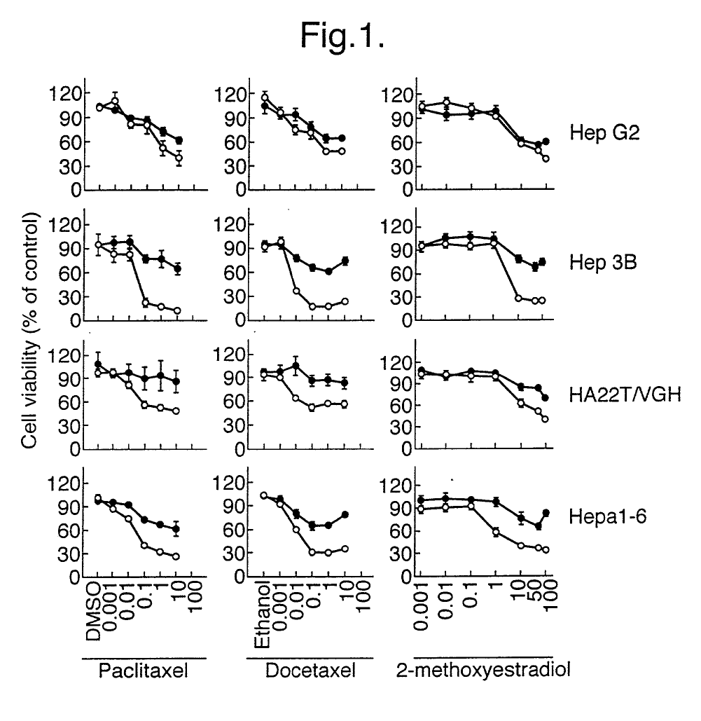

The results are shown in FIG. 1, in which filled circles represent data following treatment for 24 hours and open circles represent data following treatment for 72 hours. Data are the mean ± standard error of mean from duplicate samples of three independent experiments.

Propidium Iodide (PI) Exclusion Assay

Cells were grown on 5-cm² flasks (CORNING) and treated with paclitaxel and docetaxel as set out above. Propidium iodide (10 μg/ml) was then added for 15 minutes incubation at 37° C. Then, medium was collected prior to harvest of the adherent cells. Both suspended and attached cells were collected and resuspended with 500 μl PBS for flow cytometry analysis as set out below. Debris signals were removed by FSC-SSC gating.

Flow Cytometric Analysis of DNA Content

Lysing buffer (0.5% Triton X-100, 0.2 μg/ml Na₀₂EDTA.2H₂O, and 1% bovine serum albumin in PBS) was added to the cell pellets which were then left on ice for 15 minutes. 100% methanol pre-cooled to −20° C. was then added to the mixture, which was then centrifuged at 300 xg for 5 minutes. The supernatant was discarded and the cell pellet was washed with PBS. The washed pellet was stained with a DNA staining solution (50 μg/ml propidium iodide, and 5 kunitz/ml of RNase A) for 30 minutes at 4° C. in the dark. The DNA content of each cell was measured using a Becton Dickinson FACSCalibur flow cytometer as set out below.

Flow Cytometry

Cells (10000) were analyzed on a Becton Dickinson FACSCalibur flow cytometer using an argon-ion laser (15 mWatt) with incident beam at 488 nm. For PI exclusion assay, red fluorescence was collected through a 585 nm filter and the cell debris signals were removed by FSC-SSC gating. Data were acquired and analyzed using FACS/CELLQuest software on a Power Macintosh 7600/120 computer. Apoptotic cells and cells at specific cycle phases were determined by ModFit LT software.

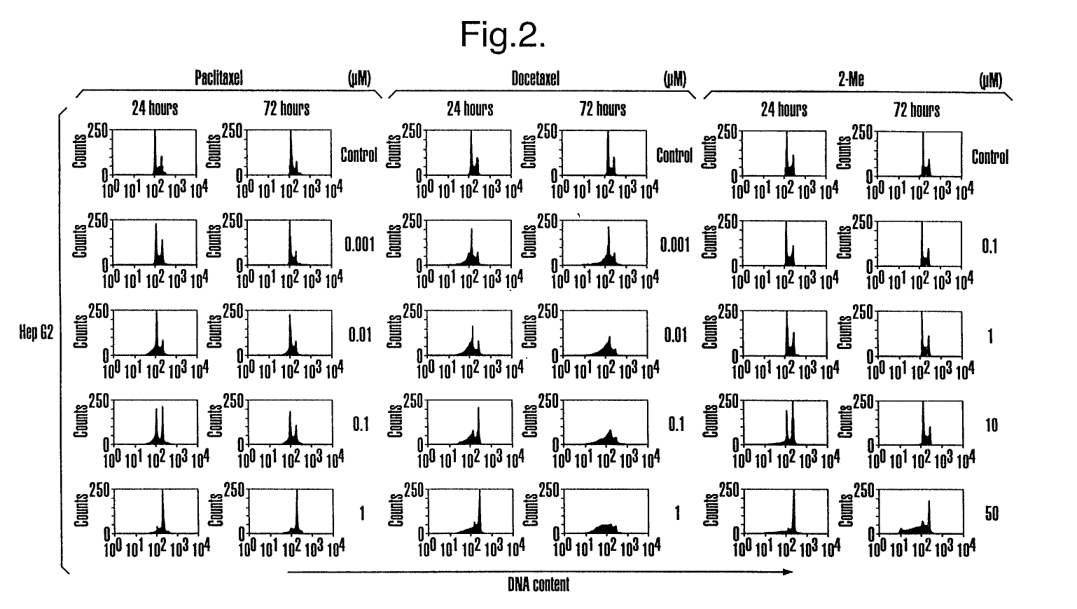

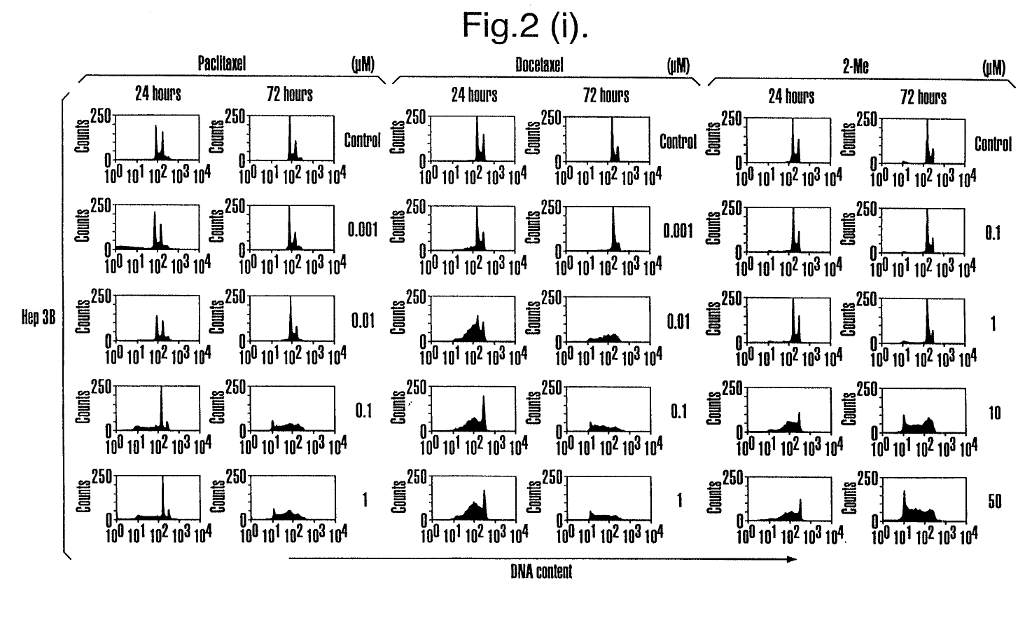

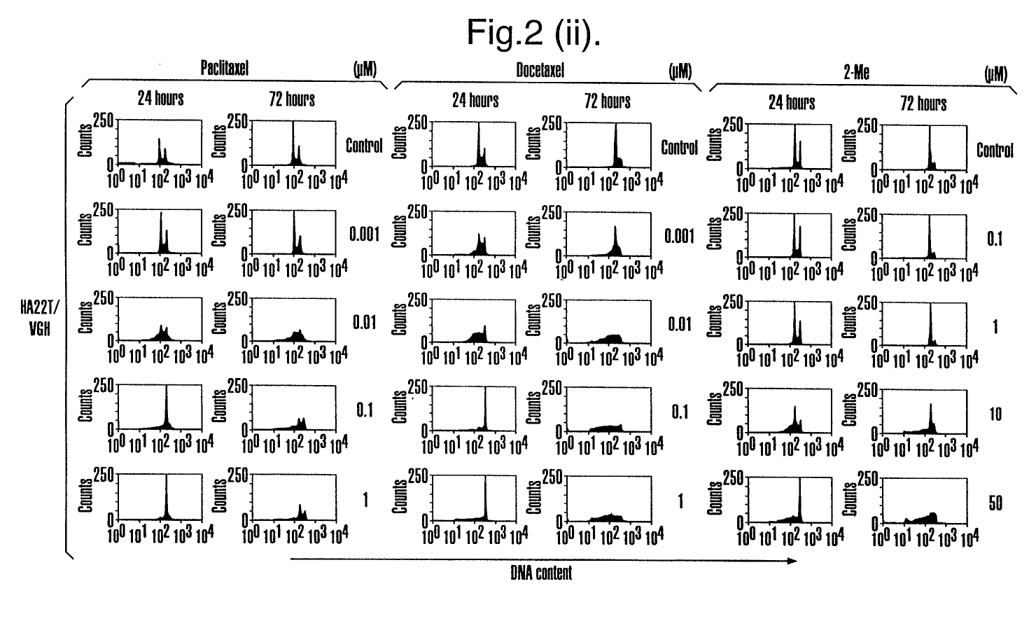

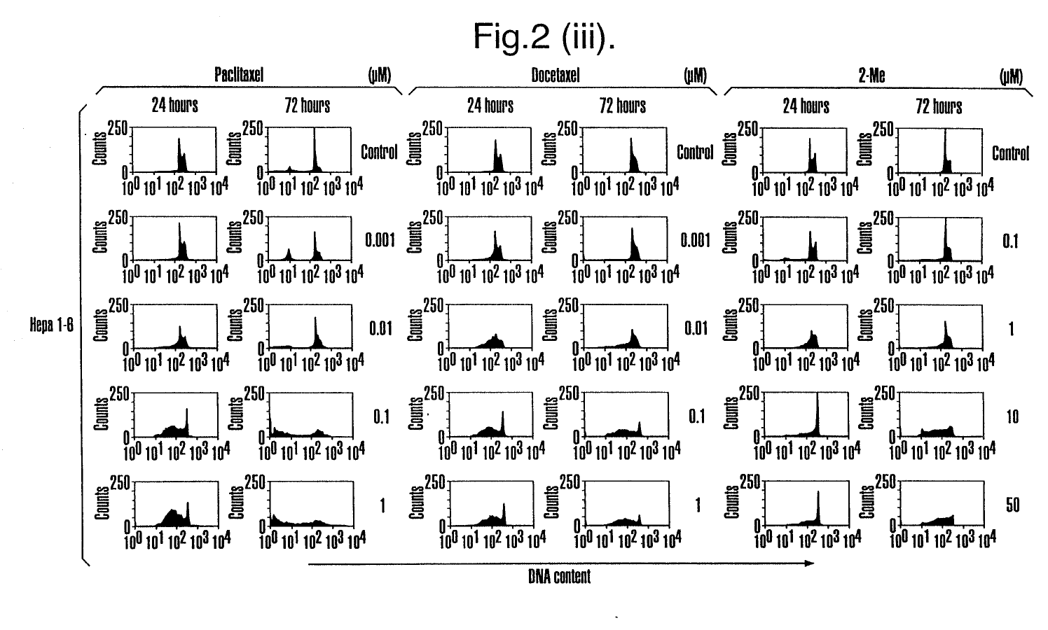

The results of the flow cytometry are shown in Tables 1 and 2 and in FIG. 2. Table 1 gives figures for cell membrane permeability of the hepatoma cells, following treatment with paclitaxel and docetaxel. Table 2 details the percentage of apoptotic (sub-G0/G1) cells found after paclitaxel and docetaxel treatment. FIG. 2 shows a DNA histogram analysis detailing the effect of paclitaxel and docetaxel on cell cycle progression.

TABLE 1
Cell membrane permeability of hepatoma cells after treatment with paclitaxel and docetaxel.

| | Paclitaxel (μM) | | | Docetaxel (μM) | | |
|---|---|---|---|---|---|---|
| | 0.01 | 0.1 | 1 | 0.01 | 0.1 | 1 |
| Hep G2 | | | | | | |
| 24 hrs | 93.63 ± 1.1 | 85.71 ± 6.8 | 66.71 ± 7.2 | 94.86 ± 1.3 | 85.49 ± 1.2 | 81.24 ± 3.2 |
| 72 hrs | 56.58 ± 28.7 | 43.79 ± 11.7 | 13.27 ± 4.3 | 61.06 ± 9.6 | 40.03 ± 9.0 | 27.42 ± 8.8 |
| Hep 3B | | | | | | |
| 24 hrs | 77.35 ± 11.7 | 63.50 ± 4.0 | 52.28 ± 4.1 | 93.80 ± 10.7 | 57.41 ± 6.8 | 57.39 ± 4.3 |
| 72 hrs | 57.00 ± 7.9 | 8.09 ± 2.3 | 1.90 ± 0.3 | 36.81 ± 14.7 | 36.25 ± 13.5 | 20.25 ± 14.4 |
| HA22T/VGH | | | | | | |
| 24 hrs | 94.08 ± 18.6 | 40.03 ± 7.8 | 34.24 ± 8.3 | 98.66 ± 9.0 | 38.71 ± 11.2 | 40.79 ± 5.0 |
| 72 hrs | 92.58 ± 21.3 | 93.38 ± 32.5 | 49.32 ± 8.3 | 55.44 ± 5.6 | 21.24 ± 0.4 | 22.03 ± 3.1 |
| Hepa 1-6 | | | | | | |
| 24 hrs | 93.17 ± 3.8 | 67.20 ± 4.4 | 62.65 ± 7.6 | 94.45 ± 1.9 | 83.35 ± 7.2 | 81.88 ± 8.7 |
| 72 hrs | 62.95 ± 5.6 | 27.79 ± 1.3 | 15.51 ± 1.0 | 77.18 ± 1.4 | 43.94 ± 3.4 | 38.90 ± 4.2 |

Data are mean ± standard error of mean from duplicated samples of at least three independent experiments. Cells were treated with drugs for 24-72 hours, and membrane permeability was measured by flowcytometric analysis of propidium exclusion in viable hepatoma cells. Data are the percentage of cells with intact cell membrane as compared to control.

TABLE 2
Paclitaxel and Docetaxel induced apoptosis

| | | Paclitaxel (mM) | | | Docetaxel (mM) | | |
|---|---|---|---|---|---|---|---|
| | | 0.01 | 0.1 | 1 | 0.01 | 0.1 | 1 |
| Hep G2 | 24 hrs | | | | 45.24 | 38.77 | 28.33 |
| | 72 hrs | | | | 42.45 | 42.44 | 56.66 |
| Hep 3B | 24 hrs | | | | 59.14 | 58.67 | 65.74 |
| | 72 hrs | | 38.37 | 47.01 | 64.12 | 81.66 | 79.33 |
| HA22T/ | 24 hrs | | | | 41.75 | 18.61 | 22.94 |
| VGH | 72 hrs | | 0 | 0 | 56.64 | 58.61 | 60.98 |
| Hepa 1-6 | 24 hrs | 24.02 | 55.64 | 64.38 | 52.81 | 50.76 | 53.80 |
| | 72 hrs | | N/A | N/A | 31.25 | 53.95 | 62.49 |

Figures are % of apoptotic (sub-G0/G1) cells as determined by flow cytometry. DNA Fragmentation Electrophoresis Analysis

DNA fragmentation assessment was according to the method of Herrmann et al, Nucleic Acids Res., 22, 5506-5507, 1994.

Briefly, HEP G2 cells (2×10⁷) were treated for 72 hours with paclitaxel and docetaxel as set out above and centrifuged. The thus obtained cell pellets were resuspended with NP-40 lysis buffer (1% NP-40 in 20 mM EDTA, 50 mM Tris-HCl, pH 7.5). After lysis of cells for a few seconds, the supernatants were collected (5 minutes at 1600 x g). The extraction was repeated with the same lysis buffer. SDS (final concentration 1%) and RNase were added (final concentration 2.5 μg/μl) to supernatants and incubated for 2 hours at 56° C. followed by digestion with proteinase K (2.5 μg/μl) for 2 hours at 37° C. Then, the mixtures were added to 10 M ammonium acetate prior to 100% ethanol precipitation for 30 minutes at -20° C. The DNA was collected by centrifiguration (10 min at 12000 x g) followed by electrophoresis on 1.5% agarose gel.

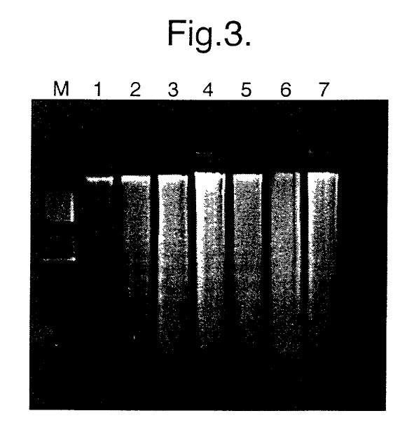

The results are shown in FIG. 3. In FIG. 3, M is a 100 base pair marker. Lane 1 shows medium control. Lanes 2 and 3 show mean paclitaxel (0.1 and 1 μM) treatment groups. Lanes 4 and 5 show mean docetaxel (0.1 and 1 μM) treatment groups.

Results

Cell Viability Studies

FIG. 1 shows the dose-dependent effect of paclitaxel and docetaxel on cell viability in hepatoma cell lines (Hep G2, Hep 3B, HA22T/VGH and Hepa 1-6). As is evident from FIG. 1, docetaxel achieved a decreased viability at 0.01 and 0.μM in nearly every case.

In Hep G2 cells, cell viability showed a decreasing trend after treatment with paclitaxel or docetaxel. The viability of Hep G2 cells was 61.81% and 39.45% of control for paclitaxel (10 μM) groups at 24 and 72 hours, respectively. For docetaxel treated Hep G2 cells, maximal reduction of viability was observed at 1 μM docetaxel, no further decrease in viability was found at 10M docetaxel. The viability was 65.03% and 48.99% for 1 μM docetaxel treated cells at 24 and 72 hours, respectively.

In Hep 3B cells it is noteworthy that significant reduction of viability (37.06%) was observed after 0.01 μM docetaxel treatment for 72 hours.

In docetaxel treated Hepa 1-6 cells, maximum cytotoxicity (65.34% and 30.71%) was found at 1 μM docetaxel treatment groups at 24 and 72 hours, respectively.

Propidium Iodide (PI) Exclusion Assay

Table 1 shows that membrane permeability of Hep G2 cells and Hep 3B cells following treatment with paclitaxel and docetaxel was dose and time dependent.

For HA22T/VGH cells, less increase in membrane permeability was observed after paclitaxel (0.01-1 μM) treatment for 72 hours as compared to that of docetaxel groups. It is noteworthy that only 55.44% of cells had intact membranes after 0.01 μM docetaxel treatment for 72 hours, whereas, following the same dose of paclitaxel, 92.58% of treated cells had intact membranes.

Cell Cycle Analysis

FIG. 2 shows that 1 μM paclitaxel-treated Hep G2 cells for 24 hours resulted in an obvious G2/M phase arrest. Similar DNA histograms were observed at 72 hours after exposure.

As shown in Table 2, apoptotic cells (sub-G0/G1) were found after treatment with 0.001 μM, 0.01 μM, 0.1 μM and 1 μM docetaxel for 24 hours with apoptotic percentages of 31.02%, 45.24%, 38.77% and 28.33%, respectively. At 72 hours after docetaxel treatment (0.001-1 μM), the apoptotic percentages were 21.92%, 42.45%, 42.44% and 56.66%, respectively.

In Hep 3B cells, 0.1 μM or 1 μM paclitaxel treatment for 24 hours resulted in G2/M arrest and incubation with 0.1 μM or 1 μM paclitaxel for 72 hours resulted in increased sub-G0/G1 percentages to 38.37% or 47.01%, respectively. In contrast, 0.01 μM, 0.1 μM or 1 μM docetaxel treated Hep 3B cells for 24 hours or 72 hours gave rise to high levels of sub-G0/G1 populations of 59.14%, 58.69% and 65.74% at 24 hours and 64.12%, 81.66% and 79.33% at 72 hours.

In HA22T/VGH cells, the increasing concentrations of paclitaxel (0.001 μM to 1 μM) correlated with the elevated percentage of G2/M cells at 24 hours. No significant sub-G0/G1 population was observed in 0.1 μM or 1 μM paclitaxel treatment groups at 72 hours. In contrast, it is significant that 0.01 μM docetaxel-treated HA22T/VGH cells at 24 hours had a higher sub G0/G1 percentage (41.75 %) than 0.1 μM (18.61 %) or 1 μM (22.94%) docetaxel groups. When cells were treated with docetaxel for 72 hours, significant sub G0/G1 percentages were found in 0.01 μM (56.64 %), 0.1 μM (58.61 %) and 1 μM (60.98 %) docetaxel-treated HA22T/VGH cells.

For Hepa 1-6 cells, paclitaxel treatment (0.01, 0.1 or 1 μM) for 24 hours resulted in increased formation of sub-G0/G1 populations (24.02 %, 55.64 % or 64.38 %, respectively), and G2/M phase arrest was observed in 0.1 μM and 1 μM paclitaxel treatment groups. When Hepa 1-6 cells were treated with 0.1 μM and 1 ,μM paclitaxel for 72 hours, most of the cells were dead and there was no obvious cell cycle profile. Docetaxel treatment (0.01 μM, 0.1 μM and 1 μM) of Hepa 1-6 cells resulted in formation of sub-G0/G1 cells (52.81 %, 50.76 % and 53.8 % at 24 hours and 31.25 %, 53.95 % and 62.49 % at 72 hours, respectively).

DNA Fragmentation Analysis

FIG. 3 shows that paclitaxel (0.1 and 1 μM) and docetaxel (0.1 and 1 μM) treatment induced DNA fragmentation in Hep G2 cells.

1. A composition for use in the treatment of hepatocellular carcinoma comprising, as active ingredient, docetaxel or a hydrate thereof.

2. A composition according to claim 1, wherein the hydrate is the trihydrate.

3. A composition according to claim 1 or 2, which is for parenteral administration.

4. A composition according to claim 3, which is suitable for intraperitoneal, subcutaneous, intravenous, intramuscular or intrasternal administration and contains from 38 to 42 mg/ml of active compound.

5. A composition according to claim 3, which is suitable for administration by infusion and contains from 0.1 to 11 mg/ml of active compound.
